(19) United States
(12) Patent Application Publication
(10) Pub. No.: US 2025/0299777 A1
(43) Pub. Date: Sep. 25, 2025

(54) SYSTEMS AND METHODS OF PHENOTYPE CLASSIFICATION USING SHOTGUN ANALYSIS OF NANOPORE SIGNALS

(71) Applicant: University of Washington, Seattle, WA (US)

(72) Inventor: Jeffrey Matthew Nivala, Seattle, WA (US)

(73) Assignee: University of Washington, Seattle, WA (US)

(21) Appl. No.: 18/862,850

(22) PCT Filed: May 3, 2023
(86) PCT No.: PCT/US2023/020877

§ 371 Date: Nov. 4, 2024

Related U.S. Application Data
(60) Provisional application No. 63/339,032, filed on May 6, 2022.

(51) Int. Cl. G16B 30/00; G06N 3/0464; G16B 40/10

(52) U.S. Cl. CPC G16B 30/00; G06N 3/0464; G16B 40/10

(57) ABSTRACT

A computer-implemented method of phenotype classification is provided. A computing system receives a plurality of segmented events generated by a plurality of nanopores in response to a sample being applied to the plurality of nanopores, wherein each segmented event of the plurality of segmented events represents ionic current changes during a protein interaction with a nanopore of the plurality of nanopores. The computing system processes the plurality of segmented events to create at least one set of model input data. The computing system provides the at least one set of model input data as input to at least one classifier model to generate a classification of the sample. The computing system transmits the classification for presentation on a display device.

### CROSS-REFERENCE TO RELATED APPLICATION

This application claims the benefit of Provisional Application No. 63/339,032, filed May 6, 2022, the entire disclosure of which is hereby incorporated by reference herein for all purposes.

### BACKGROUND

Over the past years, significant advances in DNA sequencing and analysis have allowed us to study the human genome at scale. We now better understand the interactions between genes, effects of environmental factors on gene expression, and the effects of various mutations on the phenotype. Genes encode for proteins, but it isn't a one-to-one mapping. Due to alternative splicing and post-translational modifications (information that is not directly encoded in the genome), one gene can encode for many proteins with different functions and abundance. Thus, while the human genome includes around 20,000-25,000 genes, the human proteome exists on a much larger scale, including over a million proteins. This complicates proteomics research, as we aim to develop high-throughput yet sensitive methods.

Current proteomics research most commonly involves extracting proteins from a sample, using Mass Spectrometry (MS) to identify the proteins and characterizing their abundance as well as other properties, and finally analyzing the data. However large-scale proteomics with MS is challenging since high throughput assays can't provide single molecule sensing and sensitivity to low abundance proteins. Antibody-based immunohistochemistry assays can be used to measure protein abundance levels in a sample; however, this requires developing different antibodies for different proteins. While there have been numerous advancements in MS over the past decade to improve resolution, MS still can't provide single molecule sensing and cannot identify post-translational modifications.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a computer-implemented method of phenotype classification is provided. A computing system receives a plurality of segmented events generated by a plurality of nanopores in response to a sample being applied to the plurality of nanopores. Each segmented event of the plurality of segmented events represents ionic current changes during a protein interaction with a nanopore of the plurality of nanopores. The computing system processes the plurality of segmented events to create at least one set of model input data. The computing system provides the at least one set of model input data as input to at least one classifier model to generate a classification of the sample. The computing system transmits the classification for presentation on a display device.

In some embodiments, a non-transitory computer-readable medium having computer-executable instructions stored thereon is provided. The instructions, in response to execution by one or more processors of a computing system, cause the computing system to perform actions for phenotype classification of a sample, the actions comprising: receiving, by the computing system, a plurality of segmented events generated by a plurality of nanopores in response to the sample being applied to the plurality of nanopores, wherein each segmented event of the plurality of segmented events represents ionic current changes during a protein interaction with a nanopore of the plurality of nanopores; processing, by the computing system, the plurality of segmented events to create at least one set of model input data; providing, by the computing system, the at least one set of model input data as input to at least one classifier model to generate a classification of the sample; and transmitting, by the computing system, the classification for presentation on a display device.

In some embodiments, a system comprising a flow cell and a classification computing system is provided. The flow cell comprises a plurality of nanopores, and is configured to perform actions comprising generating a plurality of segmented events generated by the plurality of nanopores in response to a sample being applied to the plurality of nanopores. Each segmented event of the plurality of segmented events represents ionic current changes during a protein interaction with a nanopore of the plurality of nanopores. The classification computing system is configured to perform actions comprising: receiving the plurality of segmented events from the flow cell; processing the plurality of segmented events to create at least one set of model input data; providing the at least one set of model input data as input to at least one classifier model to generate a classification of the sample; and transmitting the classification for presentation on a display device.

### BRIEF DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a schematic illustration of a system for nanopore-based shotgun proteomics according to various aspects of the present disclosure.

FIG. 2 is a schematic illustration of a non-limiting example embodiment of a flow cell according to various aspects of the present disclosure.

FIG. 3 is a block diagram that illustrates aspects of a non-limiting example embodiment of a classification computing system according to various aspects of the present disclosure.

FIG. 4 is a flowchart that illustrates a non-limiting example embodiment of a method of phenotype classification according to various aspects of the present disclosure.

FIG. 5 is a flowchart that illustrates a non-limiting example embodiment of a procedure for creating at least one set of model input data and providing the model input data as input to at least one artificial neural network classifier model according to various aspects of the present disclosure.

FIG. 6 is a flowchart that illustrates a non-limiting example embodiment of a procedure for creating at least one set of model input data and providing the model input data as input to at least one clustering classifier model according to various aspects of the present disclosure.

### DETAILED DESCRIPTION

Drawing inspiration from third-generation sequencing using nanopores, more recent research has sought to explore the application of nanopores for proteomics and protein sequencing. Nanopores are nano-scale, single-molecule sensors composed of pore proteins or artificially synthesized solid-state pores embedded within an insulating membrane. Passing an ionic current through a nanopore allows us to measure the disruptions in the current as analytes in solution interact with and pass through the pore. Nanopores have been used for third-generation DNA/RNA sequencing by feeding a single strand through the pore and measuring characteristic disturbances in ionic current. The resulting signal can be decoded into a nucleotide sequence. Because many nanopores can be placed on a single sensor array with an electrode connected to each channel, this technology is highly scalable.

Given the current challenges with large-scale proteomics, there is still a need for low-cost, high-throughput assays for analyzing bulk proteomic extracts. Because of its single molecule sensitivity, increased scalability, and low cost, nanopore technology has potential to be a scalable solution for high-throughput protein analysis. We seek to explore applications of nanopore technology for analyzing the protein composition of bulk proteomic extracts derived from different tissue types.

“Top-down” proteomics techniques are disclosed herein that use nanopore sensors to analyze complex, unlabeled proteomic samples derived from whole proteome extracts. In some embodiments, the techniques include generating representative nanopore data sets on individually purified proteomes from various human tissue types. Relevant machine learning approaches were explored to classify the tissue type based on its nanopore signal data, including using convolutional neural networks or clustering models to classify protein identity against a database of the subject organisms' known proteomic sequences. The techniques disclosed herein may be used to computationally predict de novo and discriminate among the ionic current signature data set features of proteomes derived from different organisms, cell types, and disease states to enable real-time proteomic analysis in applications ranging from pathogen detection to biomarker discovery and diagnostics.

Capture events are extracted from the raw nanopore signal generated when bulk proteomic extracts derived from a tissue are placed in contact with a nanopore sensor array. Events correspond to interactions between the nanopore and a protein in different conformations and for varying durations. One approach for such analyzing this data is to classify tissue type based on the capture events. Since individual events are not tagged and thus cannot be labeled by protein type and may be un-informative, we classify using the set of capture events for a given tissue. A second approach is to map nanopore data to gene or protein expression data. For a given tissue type, we have n variable length sequences x₁, x₂, . . . , xₙ˜P, where xᵢ is the normalized signal for a capture event and P is an unknown distribution representing the tissue type from which the proteomic extracts were derived. Each tissue type also has its own gene/protein expression profile, represented by the distribution q. Given background samples from P, one goal is to learn a conditional generative model \hat{q}(y|x₁, x₂, . . . , xₙ), such that \hat{q}˜q. In some embodiments, such a model may be approximated using an artificial neural network. In some embodiments, other classifier models, such as clustering models, may be used.

The “shotgun” techniques disclosed herein for analyzing events extracted from raw nanopore signal generated from bulk proteomic extracts derived from tissue provides many benefits. For example, by not requiring specific sequence read information to be generated, resource-intensive alignments of sequence reads to a reference genome need not be performed, thus greatly reducing the amount of computing power consumed by the analysis and also greatly reducing the amount of time used for the computation. As another example, being able to derive meaningful information from bulk proteomic extracts avoids the need for complicated sample preparation, isolation, purification, or other refinement steps prior to analysis of samples.

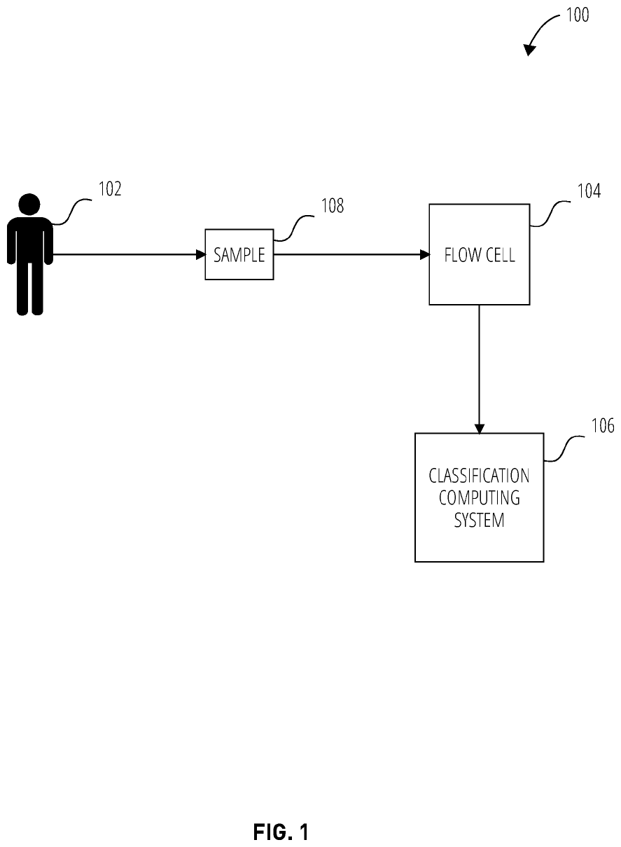

FIG. 1 is a schematic illustration of a system for nanopore-based shotgun proteomics according to various aspects of the present disclosure. As shown, a sample 108 is obtained from a subject 102 using known techniques. The sample 108 may be a tissue biopsy, a swab, a blood sample, or any other suitable type of sample 108. The sample 108 is prepared (e.g., combined with one or more buffers, enzymes, etc.), and the prepared sample 108 is provided to a flow cell 104 of a sequencing device. One non-limiting example of a sequencing device is a MinION sequencing device provided by Oxford Nanopore Technologies plc. Some non-limiting examples of devices for implementing a flow cell 104 are a Flongle Flow Cell, a MinION Flow Cell, and the PromethION Flow Cell, each also provided by Oxford Nanopore Technologies plc. The flow cell 104 generates signals based on interactions between the sample 108 and the nanopores of the flow cell 104, and provides the signals to the classification computing system 106 for analysis.

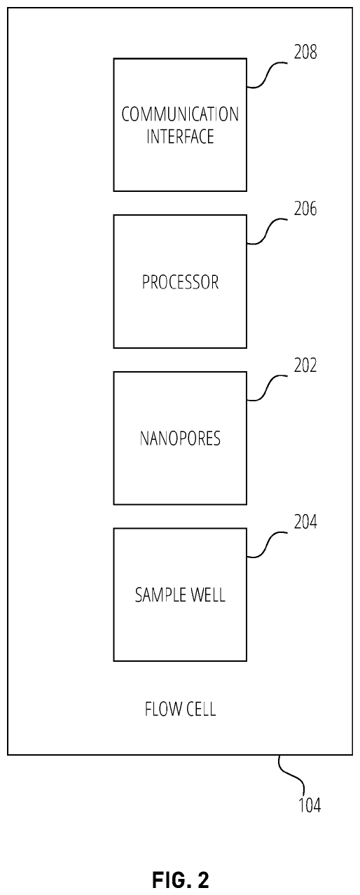

FIG. 2 is a schematic illustration of a non-limiting example embodiment of a flow cell according to various aspects of the present disclosure. As shown, the flow cell 104 includes a sample well 204, a plurality of nanopores 202, a processor 206, and a communication interface 208. The sample well 204 is configured to accept the sample 108 (e.g., to receive drops of sample 108 from a pipette) and to provide the sample 108 to the plurality of nanopores 202. The processor 206 is configured to control a voltage applied to the plurality of nanopores 202 and to read signals generated by the nanopores 202. In some embodiments, the processor 206 may also be configured to segment the signals generated by the nanopores 202 into a plurality of segmented events, each segmented event representing an interaction of a molecule with a nanopore 202 of the plurality of nanopores 202. In some embodiments, the communication interface 208 is configured to transmit the signals detected by the processor 206 to another device, such as the classification computing system 106, using a wired or wireless network, a USB connection, or any other suitable communication technique. In some embodiments, the processor 206, communication interface 208, and potentially other components (such as a computer-readable medium) may be implemented on an ASIC or FPGA that is part of the flow cell 104.

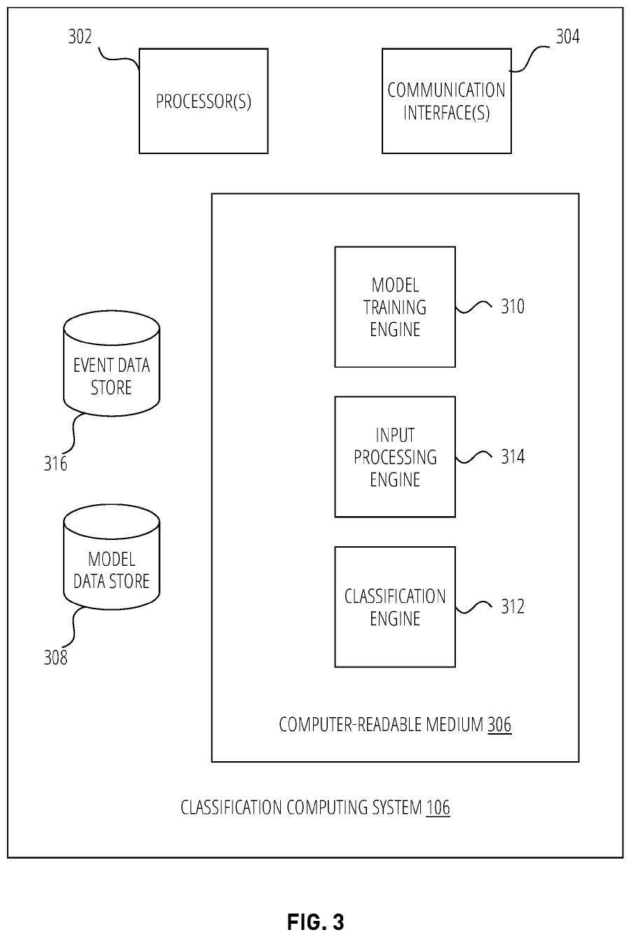

FIG. 3 is a block diagram that illustrates aspects of a non-limiting example embodiment of a classification computing system according to various aspects of the present disclosure. The illustrated classification computing system 106 may be implemented by any computing device or collection of computing devices, including but not limited to a desktop computing device, a laptop computing device, a mobile computing device, a server computing device, a computing device of a cloud computing system, and/or combinations thereof, including combinations of multiple computing devices. In some embodiments, one or more of the components illustrated as being a part of the classification computing system 106 may be provided by a flow cell or a component of a flow cell, such as an ASIC or FPGA device incorporated into the flow cell. In some embodiments, the classification computing system 106 is configured to receive segmented events generated by a plurality of nanopores and to classify the segmented events as being indicative of one or more phenotypes using one or more classifier models. In some embodiments, the classification computing system 106 is also configured to train the one or more classifier models.

As shown, the classification computing system 106 includes one or more processors 302, one or more communication interfaces 304, a model data store 308, an event data store 316, and a computer-readable medium 306.

In some embodiments, the processors 302 may include any suitable type of general-purpose computer processor. In some embodiments, the processors 302 may include one or more special-purpose computer processors or AI accelerators optimized for specific computing tasks, including but not limited to graphical processing units (GPUs), vision processing units (VPTs), and tensor processing units (TPUs). In some embodiments, the processors 302 may include one or more ASICs, FPGAs, and/or other customized computing hardware.

In some embodiments, the communication interfaces 304 include one or more hardware and or software interfaces suitable for providing communication links between components. The communication interfaces 304 may support one or more wired communication technologies (including but not limited to Ethernet, FireWire, and USB), one or more wireless communication technologies (including but not limited to Wi-Fi, WiMAX, Bluetooth, 2G, 3G, 4G, 5G, and LTE), and/or combinations thereof.

As shown, the computer-readable medium 306 has stored thereon logic that, in response to execution by the one or more processors 302, cause the classification computing system 106 to provide a model training engine 310, an input processing engine 314, and a classification engine 312.

As used herein, “computer-readable medium” refers to a removable or nonremovable device that implements any technology capable of storing information in a volatile or non-volatile manner to be read by a processor of a computing device, including but not limited to: a hard drive; a flash memory; a solid state drive; random-access memory (RAM); read-only memory (ROM); a CD-ROM, a DVD, or other disk storage; a magnetic cassette; a magnetic tape; and a magnetic disk storage.

In some embodiments, the model training engine 310 is configured to train one or more classifier models based on segmented events generated by processing samples having known phenotypes, and to store the trained classifier models in the model data store 308. In some embodiments, the input processing engine 314 is configured to obtain segmented events generated by a plurality of nanopores and to prepare them for use as input to the classifier models. In some embodiments, the input processing engine 314 may receive the segmented events while they are being generated by the flow cell. In some embodiments, the input processing engine 314 may retrieve the segmented events from the event data store 316. In some embodiments, the classification engine 312 may retrieve one or more appropriate classifier models from the model data store 308, and may provide the processed segmented events from the input processing engine 314 to the one or more classifier models to generate classifications for the sample used to generate the segmented events, and may transmit the classifications for presentation on a display device or for storage.

Further description of the configuration of each of these components is provided below.

As used herein, “engine” refers to logic embodied in hardware or software instructions, which can be written in one or more programming languages, including but not limited to C, C++, C #, COBOL, JAVA™, PHP, Perl, HTML, CSS, Javascript, VBScript, ASPX, Go, and Python. An engine may be compiled into executable programs or written in interpreted programming languages. Software engines may be callable from other engines or from themselves. Generally, the engines described herein refer to logical modules that can be merged with other engines, or can be divided into sub-engines. The engines can be implemented by logic stored in any type of computer-readable medium or computer storage device and be stored on and executed by one or more general purpose computers, thus creating a special purpose computer configured to provide the engine or the functionality thereof. The engines can be implemented by logic programmed into an application-specific integrated circuit (ASIC), a field-programmable gate array (FPGA), or another hardware device.

As used herein, “data store” refers to any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure.

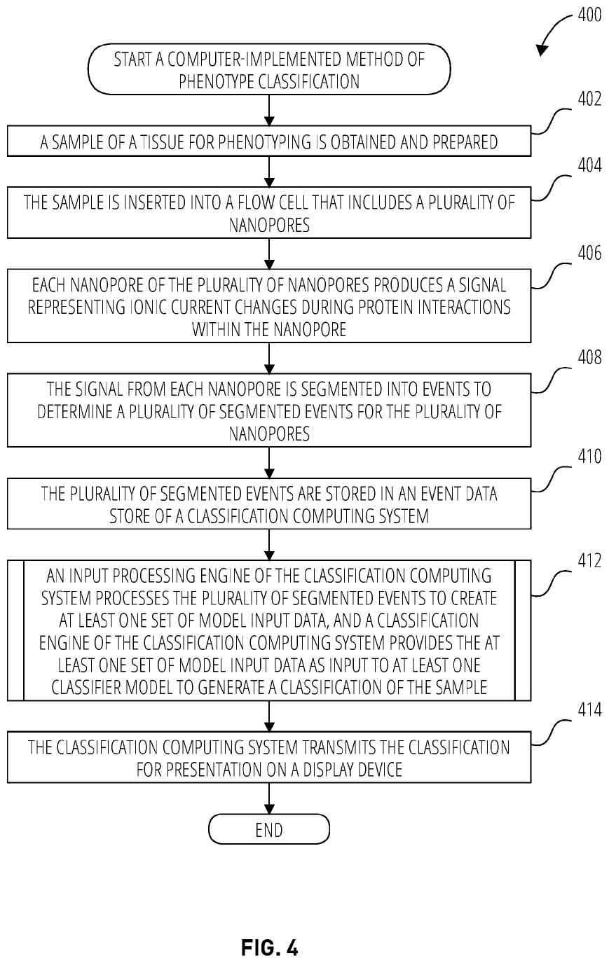

FIG. 4 is a flowchart that illustrates a non-limiting example embodiment of a method of phenotype classification according to various aspects of the present disclosure. In the method 400, raw nanopore signals generated in response to sensing a sample derived from a whole proteome extract are classified by one or more classifier models in order to determine a phenotype associated with the sample. Instead of basecalling or other complex analysis of the raw nanopore signals, segmented events of the raw nanopore signals are merely processed into a form suitable for input to the classifier models. This protein-identity-agnostic technique greatly reduces the complexity of the processing of the signals and reduces the amount of time needed for the classification compared to previous phenotyping techniques.

From a start block, the method 400 proceeds to block 402, where a sample 108 of a tissue for phenotyping (such as a tissue from a subject 102) is obtained and prepared. At block 404, the sample 108 is applied to a sample well 204 of a flow cell 104 that includes a plurality of nanopores 202. At block 406, each nanopore of the plurality of nanopores 202 produces a signal representing ionic current changes during protein interactions within the nanopore, and at block 408, the signal from each nanopore is segmented into events to determine a plurality of segmented events for the plurality of nanopores 202. The actions of block 402 through block 408 are typical for nanopore analysis of samples and are known to those of ordinary skill in the art, and are not described further herein for the sake of brevity. That said, one will note that detailed preparation of the sample 108 to prepare the sample 108 for sequencing is not performed, because raw nanopore signals in the form of segmented events will be used by the classification computing system 106 without basecalling or other sequencing-related processing.

At block 410, the plurality of segmented events are stored in an event data store 316 of a classification computing system 106. By storing the plurality of segmented events in the event data store 316, multiple different classification runs may be performed on the same plurality of segmented events, which may be useful for training the classifier models, for adjusting/comparing hyperparameters, and/or for other reasons. Further, storing the plurality of segmented events in the event data store 316 allows different computing devices to be used to process the same plurality of segmented events, if desired.

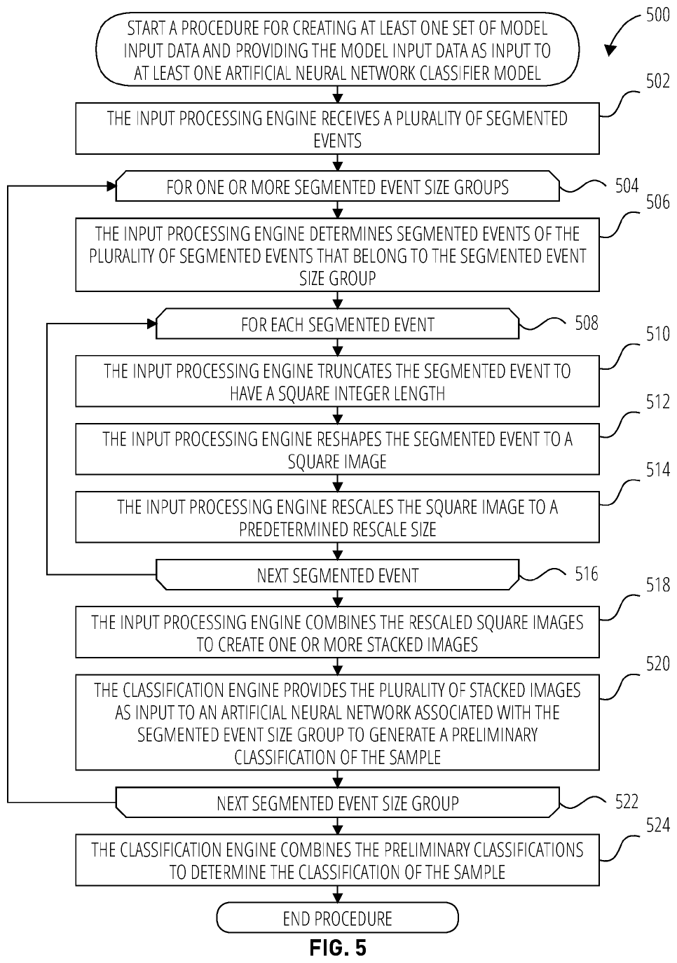

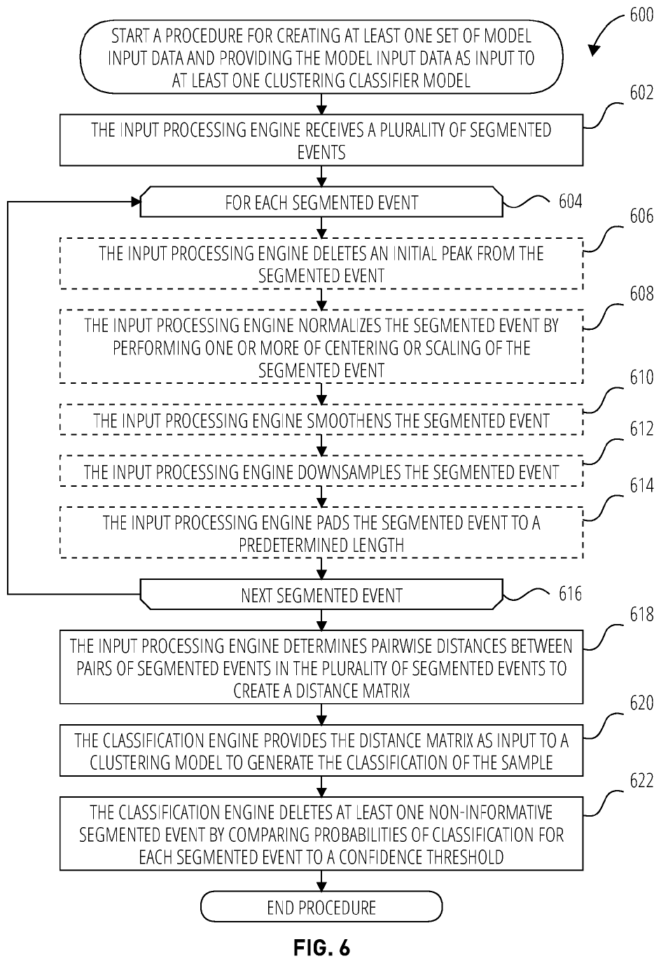

The method 400 then advances to a subroutine block 412, where a subroutine is executed wherein an input processing engine 314 of the classification computing system 106 processes the plurality of segmented events to create at least one set of model input data, and a classification engine 312 of the classification computing system 106 provides the at least one set of model input data as input to at least one classifier model to generate a classification of the sample 108. Any suitable technique may be used to create the at least one set of model input data, and any suitable classifier model (or classifier models) may be used to generate the classification of the sample 108. Typically, a technique for creating model input data will be paired with a classifier model configured to accept the type of model input data. The present disclosure includes two non-limiting examples: a technique that uses artificial neural network classifier models and an accompanying model input data creation technique (FIG. 5), and a technique that uses clustering classifier models and an accompanying model input data creation technique (FIG. 6). In some embodiments, other techniques may be used.

At block 414, the classification computing system 106 transmits the classification for presentation on a display device. The display device can be any type of display component configured to display data. As a non-limiting example, the display can include a touchscreen display. As another non-limiting example, the display can include a flat-panel display, including but not limited to a liquid-crystal display (LCD) or a light-emitting diode (LED) display. In some embodiments, the classification computing system 106 may store the classification or transmit the classification for storage. The stored classification may be used for any purpose, including but not limited to as part of a data set for re-training the classifier models, as an update to an electronic medical record, as part of a data set for research relating to the detected phenotype, or any other purpose.

The method 400 then advances to an end block and terminates.

FIG. 5 is a flowchart that illustrates a non-limiting example embodiment of a procedure for creating at least one set of model input data and providing the model input data as input to at least one artificial neural network classifier model according to various aspects of the present disclosure. In some embodiments, one or more convolutional neural networks are used as the classifier models, and the classification task is framed as an image classification problem: at a high level, the segmented events are converted into images, and classifications are generated using the convolutional neural network(s) as an image classification task.

From a start block, the procedure 500 advances to block 502, where the input processing engine 314 receives a plurality of segmented events. In some embodiments, the input processing engine 314 may retrieve an appropriate plurality of segmented events from the event data store 316. In some embodiments, the input processing engine 314 may receive the plurality of segmented events from the flow cell 104 as they are generated.

In some embodiments, all of the plurality of segmented events may be processed in the same way. However, it has been determined that the length of segmented events is typically not normally distributed. That is, it was discovered that there are typically a large number of long events (e.g., having a length greater than 30,000 data points) and an even larger number of short events (e.g., having a length less than 10,000 data points) with relatively few events in between. It was also discovered that while the short events outnumber the long events, the long events have a greater predictive power, and that different hyperparameters (e.g., stack depth, batch size, predetermined rescale size, as discussed further below) produce optimal results for different event lengths. Accordingly, in some embodiments, the procedure 500 divides the plurality of segmented events into multiple segmented event size groups for separate processing.

Accordingly, the procedure 500 then advances to a for-loop defined between a for-loop start block 504 and a for-loop end block 522, wherein each segmented event size group is processed to generate a classification. In embodiments wherein all of the segmented events are processed in a single group, the for-loop defined between for-loop start block 504 and for-loop end block 522 will be executed a single time, whereas in embodiments wherein multiple segmented event size groups are used, the for-loop defied between for-loop start block 504 and for-loop end block 522 will be executed once for each segmented event size group.

Accordingly, from the for-loop start block 508, the procedure 500 advances to block 506, where the input processing engine 314 determines segmented events of the plurality of segmented events that belong to the segmented event size group. In some embodiments, the input processing engine 314 may ignore segmented events having lengths that are below a low length threshold and/or segmented events having lengths that are above a high length threshold. In some embodiments, the input processing engine 314 may divide the remaining segmented events into segmented event size groups by comparing the lengths of the segmented events to one or more thresholds. For example, the input processing engine 314 may compare the lengths of the segmented events to a split threshold. If the length of a segmented event is shorter than the split threshold, the segmented event will be assigned to a first segmented event size group, and if the length of the segmented event is longer than the split threshold, the segmented event will be assigned to a second segmented event size group. In some embodiments, a split threshold within a range of 25,000 data points to 35,000 data points may be used, such as a split threshold of 30,000 data points.

The procedure 500 then advances to a for-loop defined between a for-loop start block 508 and a for-loop end block 516, wherein each segmented event of the segmented event size group is processed. From the for-loop start block 508, the procedure 500 advances to block 510, where the input processing engine 314 truncates the segmented event to have a square integer length, and at block 512, the input processing engine 314 reshapes the segmented event to a square image. Each data point of the segmented event is converted to a pixel value, and since the length of the segmented event is truncated to a square integer, the resulting image is square.

At block 514, the input processing engine 314 rescales the square image to a predetermined rescale size. In some embodiments, the predetermined rescale size is a hyperparameter that is adjusted for the segmented event size group. In some embodiments, the predetermined rescale size is predetermined based on a size of a smallest, largest, median, or other segmented event of the segmented event size group. Since the segmented event size group is likely to include segmented events of a variety of lengths, rescaling each of the square images to a predetermined rescale size allows them to match each other for stacking prior to submission to the classifier model. In tests, it was found that accuracy of the classification leveled off at a predetermined rescale size of 20 or 30, though in some embodiments, other values may be used for the predetermined rescale size.

The procedure 500 then advances to the for-loop end block 516. If any further segmented events remain to be processed in the segmented event size group, then the procedure 500 returns to for-loop start block 508 to process the next segmented event of the segmented event size group. Otherwise, if all of the segmented events of the segmented event size group have been processed, then the procedure 500 advances from for-loop end block 516 to block 518.

At block 518, the input processing engine 314 combines the rescaled square images to create one or more stacked images. In some embodiments, all of the rescaled square images from the segmented event size group may be combined into a single stacked image. In some embodiments, a number of rescaled square images indicated by a stack depth hyperparameter may be selected from the rescaled square images to create a stacked image. In tests, a stack depth in a range of 90-110, such as 100, was found to be optimal, though in some embodiments, other values may be used for the stack depth.

In some embodiments, the rescaled square images may be selected randomly from the segmented event size group before being combined into the stacked image. Each stacked image is a three-dimensional data structure having a two-dimensional image in the first two dimensions (i.e., the rescaled square image) and different two-dimensional images in the third dimension. The shape of this data structure is therefore (stack depth, predetermined rescale size, predetermined rescale size).

At block 520, the classification engine 312 provides the plurality of stacked images as input to an artificial neural network associated with the segmented event size group to generate a preliminary classification of the sample. In some embodiments, a single stacked image having a random sample of rescaled square images may be provided as the input to the artificial neural network. In some embodiments, multiple stacked images may be provided separately, and multiple preliminary classifications may be generated for a single segmented event size group. In some embodiments, the artificial neural network may be configured to receive as input multiple stacked images at a time.

Any suitable artificial neural network may be used to generate the preliminary classification of the sample. As stated above, since the problem has been framed as an image classification problem, a convolutional neural network (CNN) may be appropriate. In some embodiments, a CNN may be used that receives a stacked image as input and provides classifications that include one or more probabilities that the stacked image is associated with one or more phenotypes. In some embodiments, a CNN that includes a number of 2D convolutional layers followed by a fully connected layer and a final fully connected output layer may be used. Each 2D convolutional layer may include ReLU activation, 2D max pooling, dropout, and 2D batch normalization. In some embodiments, the batch size may be an additional hyperparameter to be associated with the segmented event size group. The fully connected layer may use a log-sigmoid activation function. The fully connected output layer may have a size that matches a number of phenotype classes to be predicted.

The procedure 500 then advances to for-loop end block 522. If more segmented event size groups remain to be processed, then the procedure 500 returns to for-loop start block 504 to process the next segmented event size group. Otherwise, the procedure 500 advances from for-loop end block 522 to block 524.

At block 524, the classification engine 312 combines the preliminary classifications to determine the classification of the sample 108. In some embodiments, the classification engine 312 may average (or otherwise combine) the probabilities indicated by the preliminary classifications to determine the classification of the sample 108. In some embodiments, the classification engine 312 may select a classification having a maximum or minimum probability to be used as the classification of the sample 108.

The procedure 500 then advances to an end block and returns control to its caller. One will note that, in the illustrated embodiment, the segmented event size groups are processed sequentially (i.e., all segmented events from a first segmented event size group are processed, and then all segmented events from a second segmented event size group are processed, and so on). This embodiment has been illustrated for the sake of clarity of the discussion. In some embodiments, the segmented events may be processed in any order, and the processing of segmented event size groups may instead be interleaved. That is, instead of pre-sorting the plurality of segmented events into segmented event size groups, appropriate actions for processing a given segmented event (e.g., the appropriate predetermined rescale size to be applied to the square image for the given segmented event at block 514, an appropriate stacked image to which the rescaled square image is to be added at block 518, and the appropriate artificial neural network at block 520) may be determined on the fly for each segmented event.

One will also note that, while the procedure 500 uses image stacking, in some embodiments, other techniques for combining the segmented events may be used. For example, in some embodiments, the images representing the segmented events may be tiled, or other image transformations that capture relationships between different parts of the event sequence may be used.

FIG. 6 is a flowchart that illustrates a non-limiting example embodiment of a procedure for creating at least one set of model input data and providing the model input data as input to at least one clustering classifier model according to various aspects of the present disclosure. In some embodiments, pairwise distances between the segmented events are determined to create a distance matrix, and the distance matrix is provided to one or more clustering models to determine a classification for the sample.

From a start block, the procedure 600 advances to block 602, where the input processing engine 314 receives a plurality of segmented events. As with the procedure 500 discussed above, in some embodiments, the input processing engine 314 may retrieve an appropriate plurality of segmented events from the event data store 316. In some embodiments, the input processing engine 314 may receive the plurality of segmented events from the flow cell 104 as they are generated.

In embodiments of the procedure 600, the length of the segmented events may be useful since the procedure 600 is based on computing the distance between signals. It had been determined that segmented events longer than 30,000 data points are more informative than shorter signals for classifying phenotypes. Accordingly, in some embodiments, the input processing engine 314 may retrieve segmented events that are longer than a low length threshold, or may filter retrieved segmented events to exclude segmented events that are shorter than the low length threshold. Any suitable value may be used for the low length threshold, including values in a range from 25,000-35,000 data points, such as 30,000 data points. In some embodiments, a high length threshold may be used as well, and the input processing engine 314 may retrieve segmented events that are shorter than a high length threshold, or may filter retrieved segmented events to exclude segmented events that are longer than a high length threshold. Any suitable value may be used for the high length threshold. For example, if a nanopore sensor produces a signal at 10 kHz, and if ionic current is inversed every ten seconds, a maximum usable length for a segmented event would be about 100,000 data points. Accordingly, values in a range from 95,000-105,000 data points, such as 100,000 data points, may be suitable for use as the high length threshold. In data generated during testing, it was found that the most abundant length of segmented events is close to 100,000 data points, and these interactions are expected to provide more information about the molecule interacting with the nanopore 202 than shorter signals.

The procedure 600 then advances to a for-loop defined between a for-loop start block 604 and a for-loop end block 616, where each segmented event of the plurality of segmented events is prepared for further processing. Each segmented event may be trimmed, downsampled, and/or otherwise processed in order to improve the performance of the classifier model as described in further detail below.

From the for-loop start block 604, the procedure 600 advances to optional block 606, where the input processing engine 314 deletes an initial peak from the segmented event. The initial peak of the segmented event is typically a remainder of a segmentation technique used to transform continuous nanopore signal data into the plurality of segmented events each representing the lecture of a peptide. While this initial peak may be informative, it may also distort the magnitude of the signal after other processing, including but not limited to normalization. Accordingly, a suitable initial peak threshold may be selected, and data points prior to the initial peak threshold may be deleted from the segmented event. Any suitable initial peak threshold may be chosen, including but not limited to initial peak thresholds in a range from 1500-2500 data points, such as 2000 data points.

At optional block 608, the input processing engine 314 normalizes the segmented event by performing one or more of centering or scaling of the segmented event. To center a segmented event, the input processing engine 314 subtracts the mean value from each of the data points, and then scales the signal so that the maximum value of the data points of the segmented event is 1 or so that the minimum value of the data points of the segmented event is −1. To scale a segmented event, the input processing engine 314 scales the signal such that the minimum data point value is 0 and the maximum data point value is 1. In some embodiments, other techniques for normalization, such as other techniques available in the pyts time series classification library for Python provided by Johann Faouzi and other contributors and made available as open source under a BSD license, may be used.

At optional block 610, the input processing engine 314 smoothens the segmented event. Any suitable smoothing technique may be used to smoothen the segmented event. In some embodiments, a Savitzky-Golay filter may be used to smoothen the shape of the segmented event. Smoothed signals lack some of the oscillations of the raw segmented event, and these oscillations may or may not be informative. Techniques that use a smoothing filter typically learn to classify segmented events based on general changes of intensity of the signal, as opposed to vibration or noise within the signal.

At optional block 612, the input processing engine 314 downsamples the segmented event. As noted above, the original segmented event may have between 30,000 and 100,000 data points. Computing distances using all of these data points may be too computationally expensive when working with a large number of segmented events. Accordingly, the input processing engine 314 may downsample the segmented event to fewer points by regularly sampling data points from the segmented event. In some embodiments, the data points sampled from the segmented event are equally spaced. Any suitable downsampling factor may be used, and the downsampling factor may be an adjustable hyperparameter. A downsampling factor selected from a range of 900-1100 may be appropriate, such as a downsampling factor of 1000. For an example downsampling factor of 1000, one data point out of every 1000 data points from the segmented event is sampled, such that a segmented event of 100,000 data points would result in a downsampled segmented event of 100 data points.

At optional block 614, the input processing engine 314 pads the segmented event to a predetermined length. To pad the segmented event, the input processing engine 314 may add predetermined integer data values to the segmented event until the segmented event reaches a predetermined size. By making all of the segmented events be the same predetermined size, certain benefits in processing may be obtained. For example, the segmented events may be represented in a two-dimensional numpy array, which may then be passed into a library such as sklearn. Padding the segmented events to be matching sizes may also help the accuracy of a distance measurement, though it may unnecessarily introduce undesirable artifacts in the distance measurement.

The procedure 600 then advances to the for-loop end block 616. If further segmented events remain to be processed, then the procedure 600 returns to for-loop start block 604 to process the next segmented event. Otherwise, if all of the segmented events have been processed, then the procedure 600 advances to block 618.

At block 618, the input processing engine 314 determines pairwise distances between pairs of segmented events in the plurality of segmented events to create a distance matrix. In some embodiments, a square distance matrix may be created by computing an upper half of the distance matrix with the pairwise distances, and then symmetrizing the distance matrix to fill the lower half. Values on the diagonal (representing a distance between a segmented event and itself) were zero. Any suitable technique for filling the distance matrix, which is a computationally expensive process due to the size of the distance matrix and the complexity of each pairwise distance measurement, may be used. In some embodiments, functions from the sklearn or scipy libraries may be used to create the distance matrix. In some embodiments, a routine that fills a large numpy array in parallel may be used in order to decrease the amount of time used to create the distance matrix.

The input processing engine 314 may compute each pairwise distance between pairs of segmented events using any suitable distance computation technique. In some embodiments, a dynamic time warping (DTW) technique may be used, which is known to those of ordinary skill in the art for measuring similarity between two temporal sequences which may vary in speed. One intuition leading to the choice of the DTW technique is that nanopore signals may be affected by differences in the speed the information is read from the peptides, making DTW suitable. While simpler distance comparisons, such as a Euclidean distance comparison that makes a point-to-point comparison, could be used, DTW may be preferable because it develops a one-to-many match between points. In this way, similar patterns on different time scales would correctly be determined to have a small distance between them.

The DTW computation includes computing a distance between each pair of points between both segmented events to create a matrix-like representation of the distance between the signals. The distance is obtained by summing up the distances of the path through the matrix-like representation with smaller increases in distances. This corresponds to following a path with minimum distances starting from the beginning of the segmented event.

In some embodiments, further techniques may be used to reduce the computation time for computing the DTW distance. For example, in some embodiments, a window may be defined that limits how much time stretching is allowed between each pair of signals, commonly known as a Sakoe and Chiba technique to those of ordinary skill in the art. Using a Sakoe and Chiba technique limits the time stretching allowed in the DTW computation. Since the segmented events can't be stretched as much as in the classic technique without the Sakoe and Chiba window, the resulting DTW distance between them is larger, but the computing time is reduced. In some test embodiments of the preset disclosure, a window of 0.4 for the Sakoe and Chiba technique was used, which allows up to 40% of stretching. In some embodiments, other window sizes, including but not limited to window sizes selected from a range of 0.2-0.6 may be used. In the test embodiment, the implementation of DTW in the pyts library mentioned above was used. This library implements DTW computation in a function and allows comparison of signals of different lengths. A slightly adapted DTW computation technique that can work on a GPU and in parallel was also tested.

At block 620, the classification engine 312 provides the distance matrix as input to a clustering model to generate the classification of the sample 108. Any suitable clustering model or combination of clustering models may be used, and the clustering model(s) may be trained using any suitable technique.

Typically, the clustering model may be trained using at least one set of segmented events labeled for each phenotype for which classification is desired. In test embodiments, segmented events were obtained from samples having known phenotypes, and were labeled with the known phenotypes. For example, in a test embodiment trained to classify tissue samples as heart tissue or adrenal tissue, segmented events were obtained for four technical replicates (nanopore runs on different days) of processing heart tissue and adrenal tissue samples. For training, segmented events for three technical replicates were used, and segmented events for the fourth technical replicate were used for validation.

To prepare the segmented events for training, the number of segmented events were balanced for all classes and samples. This was done at least because it is more challenging to evaluate a binary classifier if the two classes are present in different amounts (i.e., if one category has more training examples than the other category), and to reduce the total number of segmented events to be processed due to the computational complexity. The segmented events were balanced by randomly sampling (without repetition) from each technical replicate the same amount of signals that the smallest technical replicate included. This technique included two steps: (1) balancing classes by comparing the number of segmented events for heart tissue and adrenal tissue for each technical replicate, and randomly selecting segmented events from the larger set to obtain the same number of segmented events as in the smaller set; and (2) balancing signals by comparing the number of segmented events across the technical replicates and randomly selecting segmented events from each of them that is equal to the number of segmented events in the sample with the least number of segmented events. As a result, the 8 sets of segmented events (4 technical replicates each of heart tissue and adrenal tissue processing) have the same number of segmented events. In the test example, the smallest set had 4,995 segmented events, and so all 8 of the sets of segmented events were reduced to 4,995 segmented events, for a total of 39,960 segmented events to be used.

In some embodiments, a k-nearest neighbors (kNN) clustering model may be used. A kNN clustering model uses the distance matrix to identify the k closest segmented events (the k “nearest neighbors”) to each segmented event. It then classifies the segmented event based on the labels of these k nearest neighbors. Two hyperparameters may be optimized for this technique during training. A first hyperparameter is a number of neighbors considered (k). Too many neighbors or too few neighbors reduces the accuracy of the predictor. Typically, k is an odd number to avoid having a same number of neighbors from multiple classes. A second hyperparameter is a weight assigned to neighbors, which can be either “uniform” or “distance.” When selecting “distance,” the neighbors are weighted according to its distance from the segmented event, thus the labels of neighbors that are closer are more helpful than the labels of neighbors that are farther apart. When selecting “uniform,” all neighbors have the same usefulness.

For this approach, each segmented event will find some other segmented events as neighbors forming a cluster, and that each cluster will represent a peptide in the sample or a peptidic signature. Some clusters will represent peptides exclusive to a phenotype (i.e., that are present in only one of the phenotypes being trained). Ideally, in these cases, the neighbors will all be labeled in the correct phenotype class that contains this peptide. However, some clusters will represent a peptide that is present in both phenotypes. In these cases, the neighbors will be segmented events labeled as both phenotypes.

The probability predicted for a segmented event to belong to a class or another is assigned depending on the number of neighbors of each class found by the classifier model. Ideally, in the case of clusters unique to a phenotype, the probability will be close to 0 or 1 (depending on the phenotype, in the case of a binary classifier model) reflecting that all of its neighbors are from the same class. Segmented events that are similar in both phenotypes of a binary classifier model will have neighbors from both classes and its probability will be closer to 0.5. The probabilities of the labels of the segmented events are considered when computing the area under the ROC curve (AUC). They are also used to assign each segmented event to a class or another with a threshold at P=0.5.

To tune the k and weight parameters, in the test embodiment, the data was split into four different fractions. Three replicates were used as training data, and one replicate was reserved for evaluation. The probability of each signal in the evaluation set belonging to one phenotype or the other is obtained from the labels of its neighbors in the training set. These probabilities were then used to compute the accuracy and area under the ROC curve (AUC) by comparing the predicted labels with the real labels of each segmented event in the evaluation set. The mean AUC and accuracy were also computed by averaging these values across the 4 different splits. This training process is repeated for each of the k values and weight parameters tested. Once ideal values for k and weight are determined, the labeled segmented events and the values for k and weight may be stored in the model data store 308 as the trained clustering model, and newly obtained segmented events may be classified using the trained clustering model. Appropriate values for k and weight will be dependent upon the characteristics of the training data obtained. In a test embodiment, the best mean accuracy was obtained with a k of 581 (accuracy=0.613, AUC=0.636), while the best mean AUC was obtained with a k of 187 (accuracy=0.608, AUC=0.638), both with uniform weights.

At block 622, the classification engine 312 deletes at least one non-informative segmented event by comparing probabilities of classification for each segmented event to a confidence threshold. In some embodiments, this may be performed while training the clustering model. In some embodiments, this may be performed on the segmented events being provided as input to the clustering model.

If all segmented events are included in the clustering model (such as the kNN clustering model described above), classification accuracy and AUC may remain low, because many clusters may belong to peptides that are in both phenotypes. Such clusters should be considered part of a third class that represents signals present in both phenotypes. The clusters would then be divided into three groups: if the probability of belonging to one phenotype or the other is close to 0 or 1, the cluster is probably exclusive of one of the phenotypes and hence is informative to identify if the analyzed sample belongs to one phenotype or the other, and may be labeled as one of those two phenotypes. Otherwise, if the probability of belonging to one phenotype or the other is closer to 0.5, then the signal is probably present in both phenotypes. In this case, the signal is non-informative to classify the sample.

Since the goal of the clustering model is to identify a phenotype of the sample 108 by analyzing the signal composition, discarding non-informative signals should improve the accuracy of the clustering model. In a test embodiment, the kNN clustering model that had been optimized for best mean accuracy was further optimized by discarding non-informative signals. The accuracy of the model increased steadily when increasing the number of signals discarded by increasing the probability threshold for retaining signals. In a test embodiment, a peak was found when retaining 2.3% of the signals, increasing the accuracy and AUC of the clustering model from 0.6 to about 0.8. In other embodiments, other numbers of signals may be retained, including amounts ranging from 2% to 10% of the signals.

Once informative signals are identified using these kNN clustering techniques, this set of informative signals may once again be analyzed, using either the kNN clustering technique, or using another technique. For example, the informative signals may be clustered using a different technique, such as a k-medoids technique. This technique is similar to k-means clustering, but selects one of the signals as the cluster center (the centroid). This is the best approximation for time series data because the centroid of each cluster can be visualized using t-SNE. This visualization can be used to confirm that the shapes of the signals are either unique for a phenotype or shared between phenotypes.

The procedure 600 then advances to an end block and returns control to its caller.

Each of optional block 606, optional block 608, optional block 610, optional block 612, and optional block 614 are illustrated as being optional, because in some embodiments, different combinations of these preprocessing activities may be performed, the preprocessing activities may be performed in different orders, or various of these preprocessing activities may not be performed at all. In testing performed on embodiments of the present disclosure, the use of centering for normalization at optional block 608 performed worse than the use of normalization by scaling, and both performed worse than not performing normalization at optional block 608. The tests without using smoothing at optional block 610 also performed more accurately than tests performed with Savitzky-Golay smoothing. The tests without padding performed slightly better than the tests with padding performed at optional block 614. Performance of the classifier models during the tests was measured by classification accuracy (the mean value) and by area under curve (AUC) (values for each evaluation split). In general, during testing of these embodiments, adding signal processing steps seemed to decrease performance of the classifier model. One possible explanation is that the signal processing steps distorted the original signals by introducing artifacts and hence decreasing the amount of valuable information contained within the signal. That said, in some embodiments, one or more types of properly tuned signal processing steps, either alone or in combination, may be performed during the procedure 600.

While the downsampling actions of optional block 612 are also illustrated as optional, downsampling is performed in many embodiments in order to reduce the amount of computing time for the pairwise distance determinations, which have a complexity of ( )(n²). In some tests of embodiments of the present disclosure, the effects of downsampling were investigated. As discussed above, downsampling to 100 data points per segmented event significantly reduces the time to compute the distance matrices, but this may be reducing the amount of information of each signal and decreasing the performance of the classifier model.

In an first test embodiment, a distance matrix of 39,960 segmented events was computed, with three-fourths of the segmented events used as a training set and one-fourth of the segmented events used for evaluation. A reduced dataset was built by sampling one of every 5 signals of the full data set used in the first test embodiment, and was hence of 7,992 segmented events. The conditions tested were (1) the full data set with a downsample rate of 1000 (leaving 100 data points per segmented event, as described above); (2) the reduced data set with the downsample rate of 1000; (3) the reduced data set with a downsample rate of 200 (leaving 500 data points per segmented event); and (4) the reduced data set with a downsample rate of 100 (leaving 1000 data points per segmented event).

Decreasing the size of the dataset from the full dataset to only one-fifth of the dataset reduced the performance of the classifier model slightly. With the reduced dataset, increasing the downsample rate (increasing the number of data points remaining in each segmented event) improved the performance of the classifier model. The performance of the classifier model using the reduced dataset with 1000 data points per signal is similar to the performance of the classifier model using the full data set having 100 points per signal, which indicates that increasing the number of data points per segmented event improves the performance of the classifier model, albeit at an increase in computing resource utilization.

With a clustering model able to distinguish informative vs. non-informative signals and determine the phenotype of the informative signals it would be possible to cluster informative signals. Such a clustering model could be used to create a real-time in-line classifier. Such a classifier would be useful in a variety of situations, including but not limited to a tumor extraction surgery. For example, in such a surgery, the medical team would like to know if the tumor has been completely removed or there are still some carcinogenic cells in the surrounding tissue. Currently, this requires the extraction of a sample of surrounding tissue and its analysis using microscopy before the surgery can proceed. With a real-time in-line classifier, the team can perform a quick tissue extraction and apply the sample to a flow cell 104 coupled to a classification computing system 106 configured with a previously trained classifier model. The classifier model will help the team decide if the surrounding tissue is clean of carcinogenic cells or they should extract a larger part of tissue to completely remove the tumor.

In this example embodiment, each segmented event generated by the flow cell 104 may be processed in real time by the classification computing system 106 using the trained classifier model. Each segmented event is compared to a library of cluster centroid signals from the trained classifier model, one of each representing a cluster, in real time. The comparison method can be a DTW distance computation, as described above, or another kind of comparison. A list of scores of the probabilities of each segmented event belonging to one cluster or another is generated, and the clinical decision may be based on those probabilities. Further, if the segmented event was assigned to a cluster as it is being read (for instance, computing the DTW distance on the go), it would be possible to identify the signal as informative or non-informative if there was a set of clusters considered non-informative, and if the signal matches any of them it would be classified as non-informative. An alternative would be having only clusters for informative signals, so that if it does not match any of them it is considered non-informative. If the segmented event is considered noninformative, the molecule would be ejected from the pore. Such approach would be helpful to speed up the analysis, as most of the signals will belong to peptides that are shared between phenotypes. It is likely that some clusters will be more uniform (composed of signals of mostly one of the phenotype) while some others might be more diverse. Uniform clusters will have more weight than diverse clusters in classifying the sample, because they offer a higher probability of the signal to belong to one but not the other phenotype. Hence, the classifier would weight how many signals there are for each cluster and the uniformity of the clusters. With a decent set of signals assigned to a cluster or another it will be possible to obtain a robust score and identify the phenotype of the sample.

The complete disclosure of all patents, patent applications, and publications, and electronically available material cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern.

The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

As used herein, “phenotype” refers to an appearance of an organism based on a multifactorial combination of genetic traits and environmental factors; a tissue type (e.g., heart tissue vs. adrenal tissue); an organism type (e.g., a strain of bacteria); or an expressed gene.

As used herein, “nanopore” refers to a pore of nanometer size used to generate ionic current changes in response to interactions with molecules present therein.

As used herein, “nucleic acid” refers to a polymer of monomer units or “residues”. The monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxymethylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA. The five-carbon sugar to which the nucleobases are attached can vary depending on the type of nucleic acid. For example, the sugar is deoxyribose in DNA and is ribose in RNA. In some instances herein, the nucleic acid residues can also be referred with respect to the nucleoside structure, such as adenosine, guanosine, 5-methyluridine, uridine, and cytidine. Moreover, alternative nomenclature for the nucleoside also includes indicating a “ribo” or deoxyrobo” prefix before the nucleobase to infer the type of five-carbon sugar. For example, “ribocytosine” as occasionally used herein is equivalent to a cytidine residue because it indicates the presence of a ribose sugar in the RNA molecule at that residue. A nucleic acid polymer can be or comprise a deoxyribonucleotide (DNA) polymer, a ribonucleotide (RNA) polymer. The nucleic acids can also be or comprise a PNA polymer, or a combination of any of the polymer types described herein (e.g., contain residues with different sugars).

As used herein, “peptide” refers to refers to natural biological or artificially manufactured short chains of amino acid monomers linked by peptide (amide) bonds. As used herein, a peptide has at least 2 amino acid repeating units.

As used herein, “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced. “Protein” can be any of various naturally occurring substances that consist of amino-acid residues joined by peptide bonds, contain the elements carbon, hydrogen, nitrogen, oxygen, usually sulfur, and occasionally other elements (such as phosphorus or iron), and include many essential biological compounds (such as enzymes, hormones, or antibodies).

As used herein, “tissue” refers to an aggregate of similar cells and cell products forming a definite kind of structural material with a specific function, in a multicellular organism.

As used herein, “organ” refers to a group of tissues in a living organism that have been adapted to perform a specific function.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, 10 when used in this application, shall refer to this application as a whole and not to any particular portions of the application.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

It will be appreciated that, although specific embodiments of the disclosure have been described herein for purposes of illustration, various modifications may be made without deviating from the spirit and scope of the disclosure. Accordingly, the disclosure is not limited except as by the claims.

### EXAMPLES

The following numbered paragraphs describe a plurality of non-limiting example embodiments of various aspects of the present disclosure.

### Example 1

A computer-implemented method of phenotype classification, the method comprising: receiving, by a computing system, a plurality of segmented events generated by a plurality of nanopores in response to a sample being applied to the plurality of nanopores, wherein each segmented event of the plurality of segmented events represents ionic current changes during a protein interaction with a nanopore of the plurality of nanopores; processing, by the computing system, the plurality of segmented events to create at least one set of model input data; providing, by the computing system, the at least one set of model input data as input to at least one classifier model to generate a classification of the sample; and transmitting, by the computing system, the classification for presentation on a display device.

### Example 2

The computer-implemented method of example 1, wherein providing the at least one set of model input data to the at least one classifier model to generate the classification of the sample includes providing the at least one set of model input data to at least one clustering model.

### Example 3

The computer-implemented method of example 2, wherein the at least one clustering model includes a k-nearest neighbors (kNN) clustering model.

### Example 4

The computer-implemented method of example 2, wherein processing the plurality of segmented events to create the at least one set of model input data includes determining pairwise distances between segmented events of the plurality of segmented events, and wherein the at least one set of model input data includes a matrix of the pairwise distances.

### Example 5

The computer-implemented method of example 4, wherein determining the pairwise distances between the segmented events of the plurality of segmented events includes using a dynamic time warping (DTW) technique.

### Example 6

The computer-implemented method of example 5, wherein using the DTW technique includes defining a window that limits how much time stretching is allowed between compared segmented events.

### Example 7

The computer-implemented method of example 2, wherein processing the plurality of segmented events to create the at least one set of model input data includes downsampling at least one of the segmented events.

### Example 8

The computer-implemented method of example 2, wherein processing the plurality of segmented events to create the at least one set of model input data includes deleting an initial peak from at least one of the segmented events.

### Example 9

The computer-implemented method of example 2, wherein the method further comprises: deleting at least one non-informative segmented event from the at least one clustering model by comparing probabilities of classifications for each segmented event to a confidence threshold.

### Example 10

The computer-implemented method of example 1, wherein providing the at least one set of model input data to the at least one classifier model to generate the classification of the sample includes providing the at least one set of model input data to at least one artificial neural network.

### Example 11

The computer-implemented method of example 10, wherein the at least one artificial neural network includes a convolutional neural network having: four 2D convolutional layers with ReLU activation, 2D max pooling, dropout, and 2D batch normalization; a fully connected layer with a log-sigmoid activation function; and a final fully connected output layer having a size matching a number of tissue type classes to be indicated.

### Example 12

The computer-implemented method of example 10, wherein processing the plurality of segmented events to create the at least one set of model input data includes: truncating each segmented event of the plurality of segmented events to have a square integer length; reshaping each segmented event of the plurality of segmented events to a square image; rescaling each square image to a predetermined rescale size; and stacking the square images to create a plurality of stacked images to be used as the at least one set of model input data.

### Example 13

The computer-implemented method of example 12, wherein processing the plurality of segmented events to create the at least one set of model input data further includes: for each segmented event: in response to determining that the segmented event is shorter than a split threshold, using a first predetermined rescale size and stacking a first corresponding square image in a first image stack; in response to determining that the segmented event is not shorter than the split threshold, using a second predetermined rescale size and stacking a second corresponding square image in a second image stack.

### Example 14

The computer-implemented method of example 13, wherein the at least one artificial neural network includes a first artificial neural network and a second artificial neural network, and wherein providing the at least one set of model input data to the at least one artificial neural network includes providing the first image stack to the first artificial neural network and providing the second image stack to the second artificial neural network.

### Example 15

The computer-implemented method of example 1, further comprising: obtaining the sample from a subject; and providing the sample to a sample well of a flow cell that includes the plurality of nanopores.

### Example 16

A non-transitory computer-readable medium having computer-executable instructions stored thereon that, in response to execution by one or more processors of a computing system, cause the computing system to perform actions for phenotype classification of a sample, the actions comprising: receiving, by the computing system, a plurality of segmented events generated by a plurality of nanopores in response to the sample being applied to the plurality of nanopores, wherein each segmented event of the plurality of segmented events represents ionic current changes during a protein interaction with a nanopore of the plurality of nanopores; processing, by the computing system, the plurality of segmented events to create at least one set of model input data; providing, by the computing system, the at least one set of model input data as input to at least one classifier model to generate a classification of the sample; and transmitting, by the computing system, the classification for presentation on a display device.

### Example 17

The non-transitory computer-readable medium of example 16, wherein providing the at least one set of model input data to the at least one classifier model to generate the classification of the sample includes providing the at least one set of model input data to a k-nearest neighbors (kNN) clustering model.

### Example 18

The non-transitory computer-readable medium of example 17, wherein processing the plurality of segmented events to create the at least one set of model input data includes determining pairwise distances between the segmented events of the plurality of segmented events using a dynamic time warping (DTW) technique, and wherein the at least one set of model input data includes a matrix of the pairwise distances.

### Example 19

The non-transitory computer-readable medium of example 16, wherein providing the at least one set of model input data to the at least one classifier model to generate the classification of the sample includes providing the at least one set of model input data to at least one convolutional neural network.

### Example 20

A system, comprising: a flow cell comprising a plurality of nanopores; and a classification computing system; wherein the flow cell is configured to perform actions comprising: generating, by the flow cell, a plurality of segmented events generated by the plurality of nanopores in response to a sample being applied to the plurality of nanopores, wherein each segmented event of the plurality of segmented events represents ionic current changes during a protein interaction with a nanopore of the plurality of nanopores; and wherein the classification computing system is configured to perform actions comprising: receiving the plurality of segmented events from the flow cell; processing the plurality of segmented events to create at least one set of model input data; providing the at least one set of model input data as input to at least one classifier model to generate a classification of the sample; and transmitting the classification for presentation on a display device.

### Example 21

A computer-implemented method of phenotype classification, the method comprising: receiving, by a computing system, a plurality of segmented events generated by a plurality of nanopores in response to a sample being applied to the plurality of nanopores, wherein each segmented event of the plurality of segmented events represents ionic current changes during a protein interaction with a nanopore of the plurality of nanopores; processing, by the computing system, the plurality of segmented events to create at least one set of model input data; providing, by the computing system, the at least one set of model input data as input to at least one classifier model to generate a classification of the sample; and transmitting, by the computing system, the classification for presentation on a display device.

### Example 22

The computer-implemented method of example 21, wherein providing the at least one set of model input data to the at least one classifier model to generate the classification of the sample includes providing the at least one set of model input data to at least one clustering model.

### Example 23

The computer-implemented method of example 22, wherein the at least one clustering model includes a k-nearest neighbors (kNN) clustering model.

### Example 24

The computer-implemented method of example 22 or 23, wherein processing the plurality of segmented events to create the at least one set of model input data includes determining pairwise distances between segmented events of the plurality of segmented events, and wherein the at least one set of model input data includes a matrix of the pairwise distances.

### Example 25

The computer-implemented method of example 24, wherein determining the pairwise distances between the segmented events of the plurality of segmented events includes using a dynamic time warping (DTW) technique.

### Example 26

The computer-implemented method of example 25, wherein using the DTW technique includes defining a window that limits how much time stretching is allowed between compared segmented events.

### Example 27

The computer-implemented method of any one of examples 22 to 26, wherein processing the plurality of segmented events to create the at least one set of model input data includes downsampling at least one of the segmented events.

### Example 28

The computer-implemented method of any one of example 22 to 27, wherein processing the plurality of segmented events to create the at least one set of model input data includes deleting an initial peak from at least one of the segmented events.

### Example 29

The computer-implemented method of any one of examples 22 to 28, wherein the method further comprises: deleting at least one non-informative segmented event from the at least one clustering model by comparing probabilities of classifications for each segmented event to a confidence threshold.

### Example 30

The computer-implemented method of example 21, wherein providing the at least one set of model input data to the at least one classifier model to generate the classification of the sample includes providing the at least one set of model input data to at least one artificial neural network.

### Example 31

The computer-implemented method of example 30, wherein the at least one artificial neural network includes a convolutional neural network having: four 2D convolutional layers with ReLU activation, 2D max pooling, dropout, and 2D batch normalization; a fully connected layer with a log-sigmoid activation function; and a final fully connected output layer having a size matching a number of tissue type classes to be indicated.

### Example 32

The computer-implemented method of example 30 or 31, wherein processing the plurality of segmented events to create the at least one set of model input data includes: truncating each segmented event of the plurality of segmented events to have a square integer length; reshaping each segmented event of the plurality of segmented events to a square image; rescaling each square image to a predetermined rescale size; and stacking the square images to create a plurality of stacked images to be used as the at least one set of model input data.

### Example 33

The computer-implemented method of example 32, wherein processing the plurality of segmented events to create the at least one set of model input data further includes: for each segmented event: in response to determining that the segmented event is shorter than a split threshold, using a first predetermined rescale size and stacking a first corresponding square image in a first image stack; in response to determining that the segmented event is not shorter than the split threshold, using a second predetermined rescale size and stacking a second corresponding square image in a second image stack.

### Example 34

The computer-implemented method of example 33, wherein the at least one artificial neural network includes a first artificial neural network and a second artificial neural network, and wherein providing the at least one set of model input data to the at least one artificial neural network includes providing the first image stack to the first artificial neural network and providing the second image stack to the second artificial neural network.

### Example 35

The computer-implemented method of any one of examples 21 to 34, further comprising: obtaining the sample from a subject; and providing the sample to a sample well of a flow cell that includes the plurality of nanopores.

### Example 36

A non-transitory computer-readable medium having computer-executable instructions stored thereon that, in response to execution by one or more processors of a computing system, cause the computing system to perform actions for phenotype classification of a sample as recited in any one of examples 21 to 35.

### Example 37

A computing system configured to perform actions for phenotype classification of a sample as recited in any one of examples 21 to 35.

### Example 38

A system, comprising: a flow cell comprising a plurality of nanopores; and a classification computing system; wherein the flow cell is configured to perform actions comprising: generating, by the flow cell, a plurality of segmented events generated by the plurality of nanopores in response to a sample being applied to the plurality of nanopores, wherein each segmented event of the plurality of segmented events represents ionic current changes during a protein interaction with a nanopore of the plurality of nanopores; and wherein the classification computing system is configured to perform actions as recited in any one of examples 21 to 35.

1. A computer-implemented method of phenotype classification, the method comprising:
receiving, by a computing system, a plurality of segmented events generated by a plurality of nanopores in response to a sample being applied to the plurality of nanopores, wherein each segmented event of the plurality of segmented events represents ionic current changes during a protein interaction with a nanopore of the plurality of nanopores;
processing, by the computing system, the plurality of segmented events to create at least one set of model input data;
providing, by the computing system, the at least one set of model input data as input to at least one classifier model to generate a classification of the sample; and
transmitting, by the computing system, the classification for presentation on a display device.

2. The computer-implemented method of claim 1, wherein providing the at least one set of model input data to the at least one classifier model to generate the classification of the sample includes providing the at least one set of model input data to at least one clustering model.

3. The computer-implemented method of claim 2, wherein the at least one clustering model includes a k-nearest neighbors (kNN) clustering model.

4. The computer-implemented method of claim 2, wherein processing the plurality of segmented events to create the at least one set of model input data includes determining pairwise distances between segmented events of the plurality of segmented events, and wherein the at least one set of model input data includes a matrix of the pairwise distances.

5. The computer-implemented method of claim 4, wherein determining the pairwise distances between the segmented events of the plurality of segmented events includes using a dynamic time warping (DTW) technique.

6. The computer-implemented method of claim 5, wherein using the DTW technique includes defining a window that limits how much time stretching is allowed between compared segmented events.

7. The computer-implemented method of claim 2, wherein processing the plurality of segmented events to create the at least one set of model input data includes downsampling at least one of the segmented events.

8. The computer-implemented method of claim 2, wherein processing the plurality of segmented events to create the at least one set of model input data includes deleting an initial peak from at least one of the segmented events.

9. The computer-implemented method of claim 2, wherein the method further comprises:
deleting at least one non-informative segmented event from the at least one clustering model by comparing probabilities of classifications for each segmented event to a confidence threshold.

10. The computer-implemented method of claim 1, wherein providing the at least one set of model input data to the at least one classifier model to generate the classification of the sample includes providing the at least one set of model input data to at least one artificial neural network.

11. The computer-implemented method of claim 10, wherein the at least one artificial neural network includes a convolutional neural network having:
four 2D convolutional layers with ReLU activation, 2D max pooling, dropout, and 2D batch normalization;
a fully connected layer with a log-sigmoid activation function; and
a final fully connected output layer having a size matching a number of tissue type classes to be indicated.

12. The computer-implemented method of claim 10, wherein processing the plurality of segmented events to create the at least one set of model input data includes:
truncating each segmented event of the plurality of segmented events to have a square integer length;
reshaping each segmented event of the plurality of segmented events to a square image;
rescaling each square image to a predetermined rescale size; and
stacking the square images to create a plurality of stacked images to be used as the at least one set of model input data.

13. The computer-implemented method of claim 12, wherein processing the plurality of segmented events to create the at least one set of model input data further includes:
for each segmented event:
in response to determining that the segmented event is shorter than a split threshold, using a first predetermined rescale size and stacking a first corresponding square image in a first image stack;
in response to determining that the segmented event is not shorter than the split threshold, using a second predetermined rescale size and stacking a second corresponding square image in a second image stack.

14. The computer-implemented method of claim 13, wherein the at least one artificial neural network includes a first artificial neural network and a second artificial neural network, and wherein providing the at least one set of model input data to the at least one artificial neural network includes providing the first image stack to the first artificial neural network and providing the second image stack to the second artificial neural network.

15. The computer-implemented method of claim 1, further comprising:
obtaining the sample from a subject; and
providing the sample to a sample well of a flow cell that includes the plurality of nanopores.

16. A non-transitory computer-readable medium having computer-executable instructions stored thereon that, in response to execution by one or more processors of a computing system, cause the computing system to perform actions for phenotype classification of a sample, the actions comprising:
receiving, by the computing system, a plurality of segmented events generated by a plurality of nanopores in response to the sample being applied to the plurality of nanopores, wherein each segmented event of the plurality of segmented events represents ionic current changes during a protein interaction with a nanopore of the plurality of nanopores;
processing, by the computing system, the plurality of segmented events to create at least one set of model input data;
providing, by the computing system, the at least one set of model input data as input to at least one classifier model to generate a classification of the sample; and
transmitting, by the computing system, the classification for presentation on a display device.

17. The non-transitory computer-readable medium of claim 16, wherein providing the at least one set of model input data to the at least one classifier model to generate the classification of the sample includes providing the at least one set of model input data to a k-nearest neighbors (kNN) clustering model.

18. The non-transitory computer-readable medium of claim 17, wherein processing the plurality of segmented events to create the at least one set of model input data includes determining pairwise distances between the segmented events of the plurality of segmented events using a dynamic time warping (DTW) technique, and wherein the at least one set of model input data includes a matrix of the pairwise distances.

19. The non-transitory computer-readable medium of claim 16, wherein providing the at least one set of model input data to the at least one classifier model to generate the classification of the sample includes providing the at least one set of model input data to at least one convolutional neural network.

20. A system, comprising:
a flow cell comprising a plurality of nanopores; and
a classification computing system;
wherein the flow cell is configured to perform actions comprising:
generating, by the flow cell, a plurality of segmented events generated by the plurality of nanopores in response to a sample being applied to the plurality of nanopores, wherein each segmented event of the plurality of segmented events represents ionic current changes during a protein interaction with a nanopore of the plurality of nanopores; and
wherein the classification computing system is configured to perform actions comprising:
receiving the plurality of segmented events from the flow cell;
processing the plurality of segmented events to create at least one set of model input data;
providing the at least one set of model input data as input to at least one classifier model to generate a classification of the sample; and
transmitting the classification for presentation on a display device.

21.-38. (canceled)
